(19) United States
(12) Patent Application Publication
(10) Pub. No.: US 2023/0381218 A1
(43) Pub. Date: Nov. 30, 2023

(54) PREBIOTIC FORMULA, DIETARY FIBER, FOOD, PHARMACEUTICAL COMPOSITION, AND APPLICATION THEREOF

(71) Applicant: BIOCEUTICAL ATTAINMENTS COMPANY LIMITED, Kaohsiung City (TW)

(72) Inventor: Yung-pin LEE, Taipei City (TW)

(73) Assignee: BIOCEUTICAL ATTAINMENTS COMPANY LIMITED, Kaohsiung City (TW)

(21) Appl. No.: 18/201,714

(22) Filed: May 24, 2023

Related U.S. Application Data
(60) Provisional application No. 63/345,095, filed on May 24, 2022.

(51) Int. Cl. A61K 31/736; A61P 1/04

(52) U.S. Cl. CPC A61K 31/736; A61P 1/04

(57) ABSTRACT

A prebiotic formula comprises glucomannan polysaccharides with a main distribution range of molecular weight at 0.6-107 kDa, said low molecular weight glucomannan polysaccharides, having β-1,4-D-glucopyranose units and β-1,4-D-mannopyranose units in backbone, and a portion of mannosyl units acetylated. The prebiotic formula aims to provide intestinal mucosal protection and intestinal immunity modulation.

### CROSS-REFERENCE TO RELATED APPLICATIONS

This non-provisional application claims priority under 35 U.S.C. § 119(a) on Patent Application No. 63/345,095 filed in U.S. on May 24, 2022, the entire contents of which are hereby incorporated by reference.

### BACKGROUND

### 1. Technical Field

The present disclosure relates to a prebiotic formula, a dietary fiber, a food and a pharmaceutical composition including the same, and an application thereof.

### 2. Related Art

Prebiotics, as a nutrient source for probiotics, have an influence on the composition of the probiotics in the intestine and confer health benefits. Among them, water-soluble polysaccharides are appropriate raw materials due to their various sources and well-established manufacturing processes. Their physicochemical properties vary by size, chemical structure, solubility, viscosity, and fermentability, each of which influences microbial competence.

The water-soluble polysaccharides that is resistant to hydrolysis by digestive enzymes is commonly selected as prebiotic formula, such as inulin, fructo-oligosaccharides, glucan, gluco-oligosaccharides, galacto-oligosaccharides, and lacto-oligosaccharides.

However, oligosaccharides are easily consumed by facultative anaerobic Lactobacilli or oxygen-tolerant anaerobic Bifidobacteria while passing through the small intestine, resulting in small intestine bacterial overgrowth and leading to digestive abnormalities. Currently, product is still few on the market in design for metabolized by obligate anaerobes in the colon.

In addition, patients often discontinue immune checkpoint blockade (ICB) therapy due to limited response to the treatment or adverse events. Fortunately, recent findings suggest that modifying the intestinal microbiota can substantially improve efficacy of ICB therapy. Moreover, intestinal toxicity, causing adverse events such as diarrhea or colitis, is necessary to find a way to overcome. Therefore, the prebiotic formula is a suitable choice.

### SUMMARY

Accordingly, the present disclosure provides a solution to the above questions. In addition, the disclosure also aims to provide a dietary fiber, a food and a pharmaceutical composition made from the prebiotic formula, and the applications.

According to one or more embodiment of the present disclosure, a prebiotic formula comprises glucomannan polysaccharides with a main distribution range of molecular weight at 0.6-107 kDa, said low molecular weight glucomannan polysaccharides, having β-1,4-D-glucopyranose units and β-1,4-D-mannopyranose units in backbone, and a portion of mannosyl units acetylated.

Moreover, the present disclosure provides a dietary fiber, a food and a pharmaceutical composition including said prebiotic formula.

Furthermore, the prebiotic formula of the present disclosure mediates the functions of intestinal mucosa protection and intestinal immunity modulation. Said prebiotic formula is fermented by inducible microbiota in the intestine after ingestion. The type of said inducible microbiota includes the clostridial IV or XIV clusters or the genera of Clostridium, Bacteroides, Alistipes, Bifidobacterium, Lactobacillus, Enterococcus or Akkermansia.

Said intestinal mucosa protection includes, by said inducible microbiota, maintaining cell density of the intestinal epithelium, reducing the aberrancy of the intestinal mucosa, maintaining or improving integrity of the intestinal mucosal barrier, or increasing the immune tolerance of the intestinal mucosa.

Said intestinal immunity modulation includes, by said inducible microbiota, stimulating the activation of innate immune cells, facilitating the development and maturation of isolated lymphoid follicles (ILFs), or elevating the number of ILFs in the intestinal mucosa.

Additionally, the disclosed prebiotic formula mediates the functions of assisting ICB therapy in improving efficacy of ICB therapy, or reducing or preventing intestinal toxicity in the therapy. Said prebiotic formula is fermented by the inducible microbiota in the intestine after ingestion so as to elevate the number and facilitate the development and maturation of ILFs to provide mature adaptive CD4⁺ T, CD8⁺ T cells for the purpose of therapy.

In view of the above description, the disclosed prebiotic formula can protect intestinal mucosa, modulate intestinal immunity, or assist ICB therapy in improving efficacy of ICB therapy, or reducing or preventing the intestinal toxicity in the therapy.

### BRIEF DESCRIPTION OF THE DRAWINGS

The present disclosure will become better understood from the detailed description given hereinbelow and the accompanying drawings which are given by way of illustration only and thus are not intended to limit the present disclosure and wherein:

FIG. 1 demonstrates the diagrams between the intensity and the retention time (Rt), obtained by gel permeation chromatography (GPC), in which (A) for disclosed prebiotic formula of an embodiment and (B) for three independent batches (indicated by solid lines) compared with the standards (S1-S6, indicated by dashed lines);

FIG. 2 illustrates a scheme for the experimental design of embodiments of the present disclosure.

FIG. 3 shows the H&E staining images of the colonic tissues from each experimental group, as well as their local magnifications.

FIG. 4 shows the immunohistochemical (IHC) staining images of Foxp3 in the colonic tissues from each experimental group.

FIG. 5 shows the staining images of CD3⁺ T cells from each experimental group, as well as their local magnifications.

FIG. 6 shows the staining images of CD4⁺ T cells from each experimental group, as well as their local magnifications.

FIG. 7 shows the IHC staining images of IL-6 from each experimental group.

FIG. 8 shows the staining images of the expression of T cells within the ILFs in the DSS group.

FIG. 9 shows the staining images of the expression of T cells within the ILFs in the Formula/DSS group.

FIG. 10 shows the statistical analysis chart of the cell density of the epithelium and the aberrancy score of the intestinal mucosa from each experimental group.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

Moreover, the verification of the experimental results in the embodiments of the present disclosure involves sacrifice of the experimental animals. In order to conform to the international “3Rs” initiative of animal welfare, the numbers of the experimental animals used in the present disclosure is limited, but it is sufficient to scientifically demonstrate the experimental results. Therefore, it should be understood that these embodiments mentioned below are not Intended to restrict the extent of the present disclosure.

### Prebiotic Formula

The disclosed prebiotic formula comprises glucomannan polysaccharides with a main distribution range of molecular weight at 0.6-107 kDa, said low molecular weight glucomannan polysaccharides, having β-1,4-D-glucopyranose units and β-1,4-D-mannopyranose units in backbone, and a portion of mannosyl units acetylated.

Natural glucomannan polysaccharides are not readily fermented in the intestine owing to high molecular weight, and artificial hydrolysis is required.

Artificial hydrolysis consisting of acidic, enzymatic, ultrasonic or microwave hydrolysis is well established. The hydrolyzed are classified as oligosaccharides (<4 kDa) having 3 to 20 sugar units, low molecular weight polysaccharides (4-200 kDa) having 20 to 1,000 sugar units, less-high molecular weight polysaccharides (<1,000 kDa) having less than sugar units, and high molecular weight polysaccharides having more than 5,000 sugar units, depending on demand.

The disclosed prebiotic formula eschews being consumed by the facultative anaerobic Lactobacilli or the oxygen-tolerant anaerobic Bifidobacterium while passing through the small intestine, which enables them to reach the colon and be metabolized by obligate anaerobes.

In addition, since linear structure in aqueous solution, the disclosed prebiotic formula is reacted with a plurality of endo-1,4-β-mannosidase simultaneously, which is advantageously fermented by the intestinal bacteria. If the polysaccharide becomes curved, folded, or tangled due to elongation, the resulting steric hindrance may hinder the reaction.

Furthermore, a portion of mannosyl units is acetylated, and thus it facilitates hydration.

### Dietary fiber

The disclosed prebiotic formula may be incorporated into a dietary fiber. In the subject, it may include other ingredients or food additives, which conform to the regulations and do not have negative influence on the effect of the prebiotic formula.

### Food

The disclosed prebiotic formula may be incorporated into a nutrient or supplement composition as food. In the subject, it may include other ingredients or food additives, which conform to the regulations and do not have negative influence on the effect of the prebiotic formula.

### Pharmaceutical Composition

The disclosed prebiotic formula may also be incorporated into a pharmaceutical composition. In the subject, it may include other additives or active ingredients, which conform to the regulations and do not have negative influence on the effect of the prebiotic formula.

### Applications

The disclosed prebiotic formula may be fermented by the inducible microbiota in the intestine after ingestion, which mediate the mechanisms of initiating intestinal mucosa protection and intestinal immunity modulation. The applications will be described in detail below.

### Inducible Microbiota

The disclosed prebiotic formula is designed on the basis of the enzymatic characteristics of the metabolisms amongst the known intestinal bacteria. The composition of the inducible microbiota has the clostridial IV or XIV clusters, or the genera of Clostridium, Bacteroides, Alistipes, Bifidobacterium, Lactobacillus, Enterococcus or Akkermansia.

For example, the disclosed prebiotic formula in the intestine may be directly detected by glycan sensor/transcriptional regulator on the surface of Bacteroides ovatus (B. ovatus) or Bacteroides thetaiotaomicron (B. thetaiotaomicron) etc., inducing specific endo-1,4-β-mannosidase on the membrane leading to the primary hydrolysis. Meanwhile, acetyl-mannan esterase and β-glucosidase are also encoded to hydrolyze the acetyl groups and the glucosyl units, respectively. Moreover, Faecalibacterium prausnitzii (F. prausnitzii) or Roseburia intestinalis (R. intestinalis) may perceive the primary hydrolysate of free sugar with 5 to 7 units, expressing exo-1,4-β-mannosidase in the cytoplasm for the secondary hydrolysis. The residual hydrolysate is mainly non-reducing-end mannotriose or mannobiose, which may be further hydrolyzed by β-mannanase and metabolized to butyric acid by Clostridium butyricum (C. butyricum) or Bifidobacterium adolescentis (B. adolescentis) and so on. The symbiotic community is dominated rapidly depending on a cascade expanding.

Moreover, the primary hydrolysate may be fermented by the resident bacteria in mucus, such as Akkermansia muciniphila (A. muciniphila) or Bifidobacterium pseudolongum (B. pseudolongum), resulting in their population growth and inosine production. Mannotriose of the secondary hydrolysate may be fermented by the genera of Alistipes. The free disaccharides or monosaccharides may be fermented by the genera of Bifidobacterium or Lactobacillus for acetic acid or lactic acid production and their population growth. They may also be fermented by the genera of Enterococcus for lactic acid production to compete the population growth against the phylum of Proteobacteria.

### Application to Intestinal Mucosa Protection

The disclosed prebiotic formula may be fermented by the inducible microbiota in the intestine to modulate the innate immunity to protect the intestinal mucosa, thereby strengthening the intestinal mucosal barrier.

The term “intestinal mucosal barrier” herein refers to the barrier formed by the collective of epithelial cells and goblet cells together with the mucus secreted from them, and

Paneth cells together with the antimicrobial peptides produced by them, and it separates the intestinal microbiota from the immune cells in the lamina propria.

Although the detailed mechanism by which the inducible microbiota impacts the intestinal mucosa remains unclear, promoting the renewal of the epithelial cells is an effective way to maintain the intestinal mucosal barrier function. Autophagy of the epithelial cells is a self-restoring process, allowing amino acids or fatty acids to undergo oxidative phosphorylation in the mitochondria, which leads to the rapid consumption of oxygen and nutrients in the cell. Moreover, under the circumstance of hypoxia in the intestinal mucosa, the inflammatory factors expressed by immune cells may be reduced, alleviating the mucosal inflammation and further triggering more autophagy of the epithelial cells.

As an example of promoting autophagy, Enterococci may avoid the attacks from the host macrophages so that they may opportunistically infect the host. As a response to Enterococci infection, if the epithelial cells are infected with Enterococci, the epithelial cells will be stimulated to activate the autophagy-related gene, inducing autophagy in the epithelial cells so as to clear the bacterial infections within the cells.

Furthermore, autophagy may also help to recycle the cellular nutrients, thereby preventing the nutrients from leaking into the mucosa and causing E. coli infection.

### Application to Intestinal Immunity Modulation

The communication between the inducible microbiota and the microfold (M) cells in the intestinal mucosa may induce the type 3 innate lymphoid cells (ILC3) to regulate the differentiation of naive CD4⁺ T cells into either pro-inflammatory helper T cells (Th17) or anti-inflammatory regulatory T cells (Tregs). By repetitive stimulation of the balance between pro- and anti-inflammatory responses, the immune tolerance may be enhanced, and the integrity of the intestinal mucosal barrier may be strengthened, thereby defending against bacterial invasion in the intestines.

Furthermore, some of the lymphocytes in the lamina propria are attracted by the inducible microbiota to migrate to the epithelium and become the intraepithelial lymphocytes (IELs), thereby involved in the renewal of the epithelial cells and the surveillance of change in the mucosal bacteria. The IELs release cytokine IL-6 to stimulate the proliferation and differentiation of the crypt stem cells in the intestinal epithelium, leading to a tight alignment of the neighboring epithelial cells and an increase in the secretion of mucus by goblet cells so as to protect the intestinal mucosa from various bacteria, food antigens, or chemical substances. As such, this protects the intestinal mucosa against acute or chronic intestinal mucosal inflammation, polyps, or tumorigenesis.

### Application to Assisting ICB Therapy

Butyrate is a metabolite from butyric acid producing bacteria such as Clostridium butyricum (C. butyricum) or Faecalibacterium prausnitzii (F. prausnitzii) among the inducible microbiota. Since there lacks glucose and oxygen in the colon, the aerobic glycolysis of the epithelial cells is restricted, thereby relying on butyrate as their primary energy source.

The epithelial cells absorb and transport butyrate to the mitochondria, implementing β-oxidation and oxidative phosphorylation to generate energy. This process consumes the last oxygen under hypoxia, creating a predominantly anaerobic environment in the intestinal mucosa. Therefore, none of oxygen can effectively diffuse into the intestinal lumen, thereby maintaining the oxygen-exhausted condition so as to promote the growth and metabolism of the obligate anaerobes.

### Formation of the Isolated Lymphoid Follicles

Under the anaerobic environment, hypoxia-inducible factor, HIF-1α, of ILC3 may be stabilized, facilitating the synthesis of the transcription factor, HIF-1, to promote the proliferation of ILC3 and express the retinoic-acid-related-orphan-receptor-gamma-t (RORγt), thereby becoming RORγt⁺ ILC3 cells.

In addition, with the communication by microfold cells, the resident mononuclear phagocytes within the lamina propria secrete IL-1β to stimulate the expression of Csf-2 (also known as GM-CSF) by RORγt⁺ ILC3 cells, promoting the proliferation per se and then differentiation into dendritic cells or macrophages. Among them, the dendritic cells secrete IL-10 to suppress inflammation and generate retinoic acid to activate RORγt⁺ ILC3 cells, thereby facilitating development of the cryptopatches and then maturation into the ILFs.

### Improving Efficacy of ICB Therapy

Since cancer cells inhibit immune checkpoints carried by cytotoxic T lymphocytes (CTLs), they cannot be recognized by CTLs generally, leading CTLs unable to trigger cytotoxic effects. Under ICB therapy, artificial monoclonal antibodies are applied to blockade the immune checkpoints, significantly increasing the sensitivity of the immune cells such as CTLs to recognize cancer cells and exert cytotoxic effects. Therefore, the quantity of the adaptive CD8⁺ T cells in the patient during ICB therapy is a factor influencing the efficacy.

The retinoic-acid-activated RORγt⁺ ILC3 cells can express the major histocompatibility complex II, and present non-specific peptides to naive CD8⁺ T cells, thereby aiding the development of the adaptive CD8⁺ T. Therefore, the ILFs may provide corresponding cells for the need in ICB therapy.

Furthermore, inosine, the metabolite produced by the inducible microbiota, may serve as nutrition for lymphocytes survived in the ILFs under glucose deprivation. This improves the metabolic adaptability of T cells so as to improve ICB therapy.

In summary, the disclosed prebiotic formula may increase the number and facilitate the development and maturation of ILFs in the intestinal mucosa to help the differentiation, development, and maturation of the adaptive CD4⁺ T, CD8⁺ T cells. As a result, improved efficacy may be obtained during ICB therapy.

### Reducing the Intestinal Toxicity

The ILFs which become mature after stimulated by the inducible microbiota may provide an environment for the differentiation, development and maturation of the adaptive CD4⁺ T, CD8⁺ T cells. In the germinal center of the mature ILFs, the number of B cells is far more than that of the resident innate T lymphocytes. B cells primarily carry out the tasks regarding memory and antigen presentation to assist the innate T cells in modulating the development of the adaptive CD4⁺ T, CD8⁺ T cells.

The resident and mature adaptive CD8⁺ T cells within the ILFs avoid triggering toxicity, and they are called memory effector T lymphocytes meanwhile. After they enter peripheral circulation and infiltrate into lymphoid tissues of tumor, they can implement cytotoxic effects after induced by ICB. As a result, intestinal toxicity may be reduced or prevented in ICB therapy.

Moreover, to assess whether an individual's response to ICB therapy is favorable or not, the type and relative abundance of the patient's fecal microbiota may be analyzed and compared to the inducible microbiota.

### EMBODIMENTS

The term “relative molecular weight” herein means the molecular weight calculated on the basis of the standards by gel permeation chromatography.

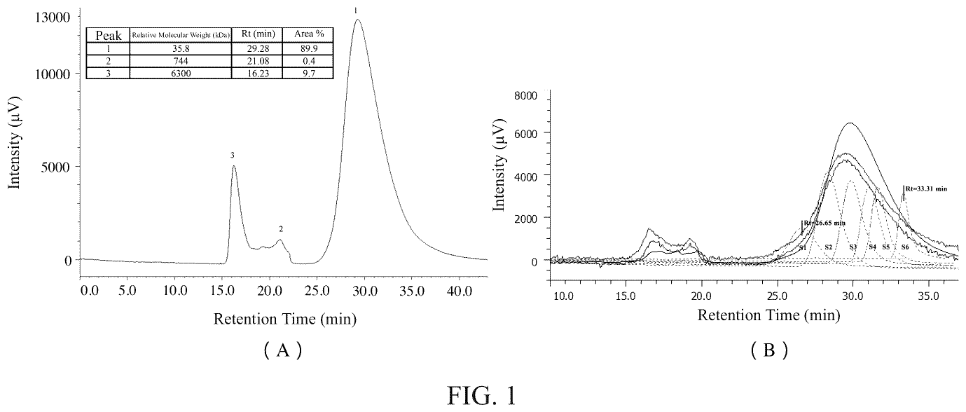

FIG. 1 demonstrates (A) the relative molecular weight of the main peak is 35.8 kDa, and another peak is 6300 kDa or 744 kDa, and (B) the main distribution range of molecular weight of the disclosed prebiotic formula is 0.6-107 kDa. Rt values of the standards may be changed following with variate of GPC condition.

Table 1 shows the molecular weight of the standards S1-S6.

| | | Retention time at peak |
|---|---|---|
| Standard | Molecular weight (kDa) | (Rt, min) |
| S1 | 107 | 26.65 |
| S2 | 47.1 | 28.30 |
| S3 | 21.1 | 30.33 |
| S4 | 9.6 | 31.47 |
| S5 | 6.1 | 32.01 |
| S6 | 0.6 | 33.31 |

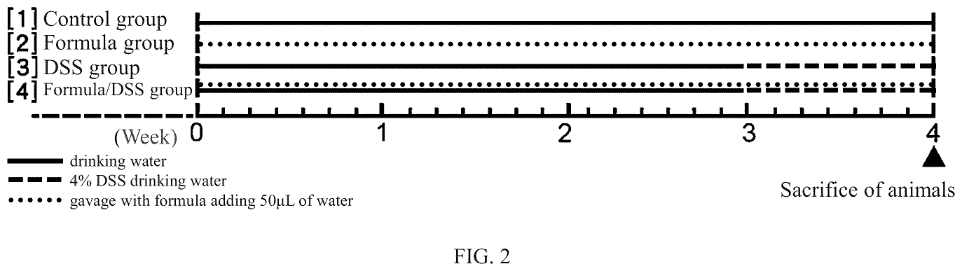

FIG. 2 illustrates a scheme for the experimental designs. After one week of cohabitation in the same cage, 12 male BALB/c mice (6-8 weeks old) are randomly divided into 4 groups, with 3 mice per group, and are then housed separately on the basis of the experimental designs. The experimental conditions for each group are as follows.
[1] Control group: mice with normal chow diet and drinking water.
[2] Formula group: mice oral gavage with the disclosed prebiotic formula in 50 μL of water and with normal chow diet and drinking water.
[3] DSS group: mice with normal chow diet and drinking water, and the drinking water is replaced with 4% DSS (dextran sulfate sodium, molecular weight about 40 kDa) aqueous solution as a pro-inflammatory agent from day 21.
[4] Formula/DSS group: mice oral gavage with the prebiotic formula of the present disclosure in 50 μL of water and with normal chow diet and drinking water, and the drinking water is replaced with 4% DSS aqueous solution from day 21.

The mice are sacrificed on day 28 and subjected to routine pathological evaluations to assess the condition of the epithelial cells and the immune cells in the colonic mucosa.

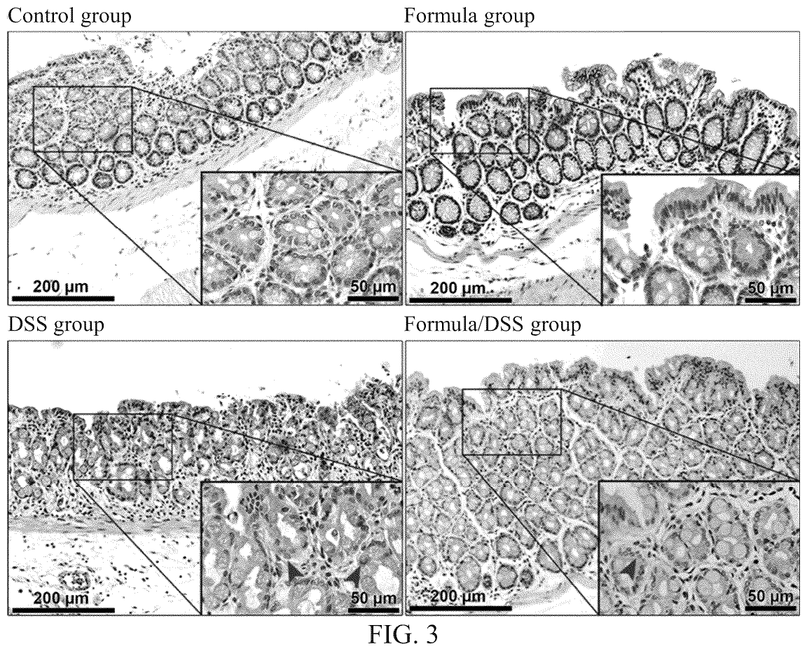

FIG. 3 shows the H&E staining images of colonic tissues from each experimental group, as well as their local magnifications. In the Control and Formula groups, healthy and intact epithelial and crypt cells are observed. In the DSS group, it is observed that the nuclei (presented by black dots) in the damaged epithelial or crypt cells become not obvious because of necrosis with folliculate enlargement caused by inflammation. In the Formula/DSS group, it is observed that the damaged epithelial or crypt cells appeared shrunken and underwent autophagy (the damaged crypt cells are indicated by arrowheads respectively). On the other hand, in the DSS group, a large amount of polymorphonuclear leukocytes enter the lamina propria of the intestinal mucosa, indicating that the intestinal mucosa tissue is infected. In contrast, in the Formula/DSS group, monocytes are predominant in the lamina propria of the intestinal mucosa, maintaining the integrity of the epithelium and crypt.

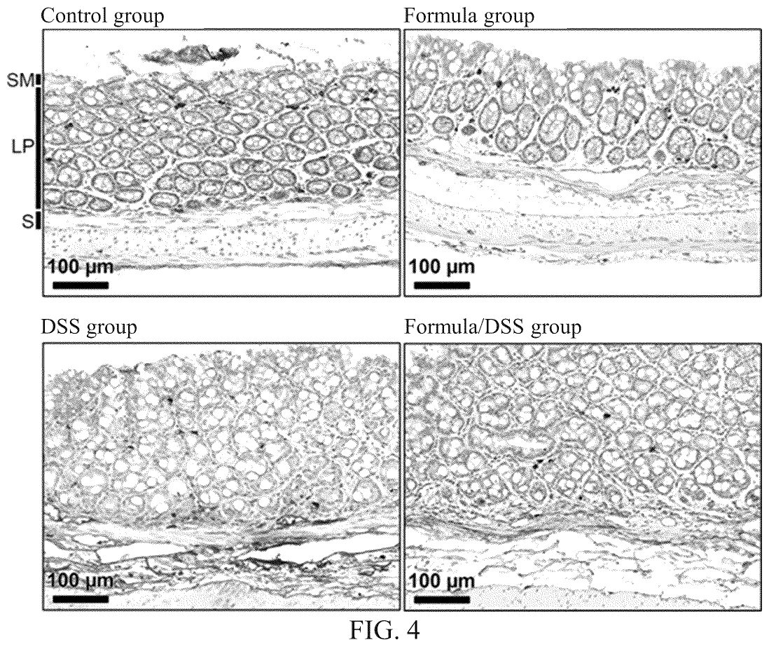

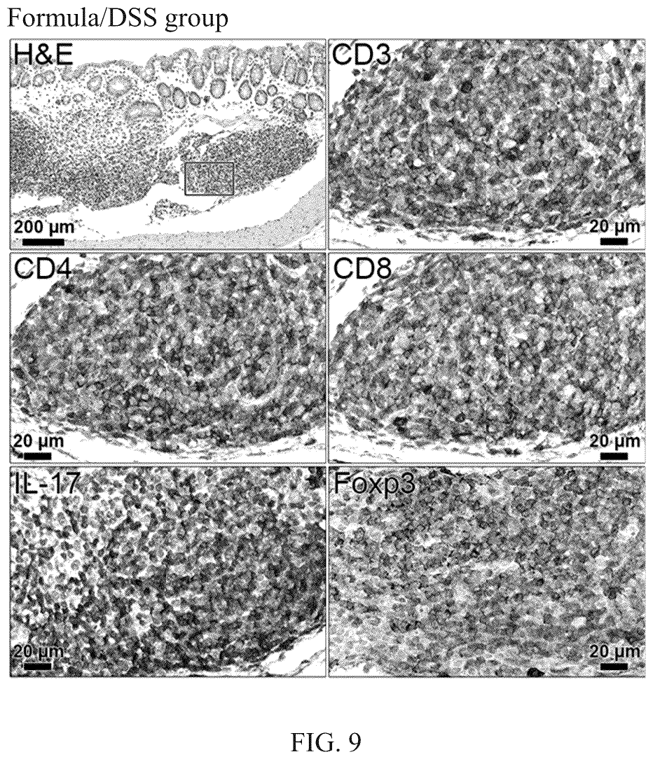

FIG. 4 shows the immunohistochemical (IHC) staining images of Foxp3 (presented by black-blurry dots) in colonic tissues from each experimental group. Compared to the Control group, the Formula group exhibits more natural regulatory T cells with the transcription factor Foxp3 distributed within the lamina propria (LP). The DSS group has significantly fewer Tregs in the lamina propria, and a large amount of the induced regulatory T cells (mainly induced by TGF-β and involved in repressing the activity of CD8⁺ T cells) distributed in the submucosa (S), which indicates that the intestinal mucosal barrier has been damaged, triggering a specific immune response caused by the infiltration of exogenous antigens into tissues. On the other hand, in the Formula/DSS group, only a few numbers of active Tregs in the lamina propria is observed, most of the Tregs cluster inside the ILFs (refer to FIG. 9), and the expression of Tregs in the submucosa is also not significant, indicating that the mature ILFs play a role in immune modulation.

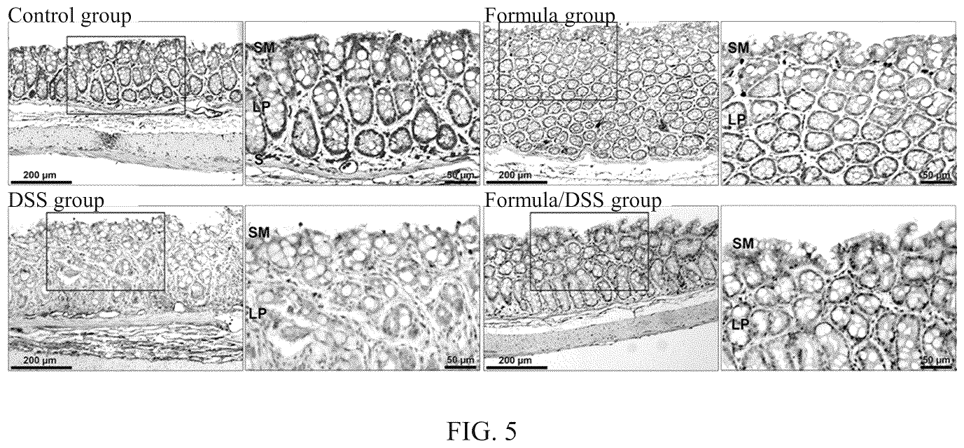

FIG. 5 shows the staining images of CD3⁺ T cells (presented by black dots) from each experimental group, as well as their local magnifications. In the Control group, CD3⁺ T cells are sporadically distributed within the lamina propria. In the Formula group, CD3⁺ T cells are dispersed below the epithelium (the junction between the superficial mucosa (SM) and the lamina propria). In the DSS group, CD3⁺ T cells cluster in the submucosa. In the Formula/DSS group, some T cells migrate into the epithelium and become intraepithelial lymphocytes, which are able to protect the mucosal epithelium.

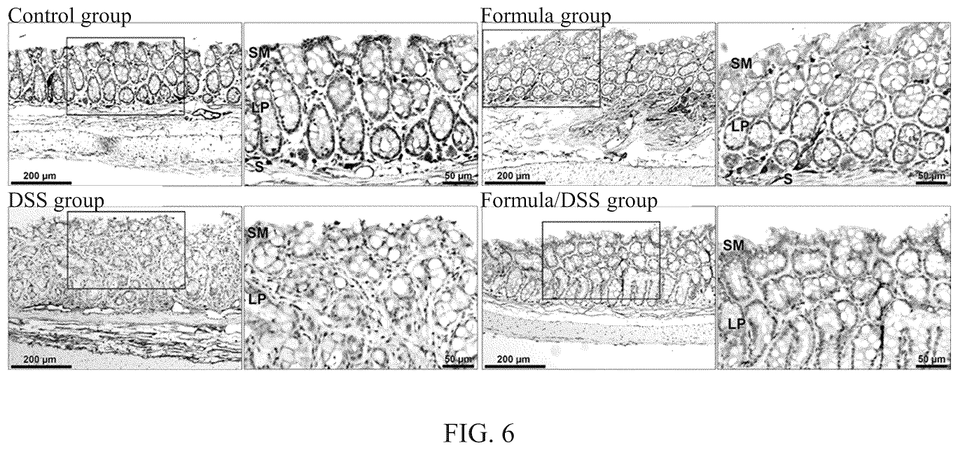

FIG. 6 shows the staining images of CD4⁺ T cells (presented by black dots) from each experimental group, as well as their local magnifications. In the Control group, CD4⁺ T cells are sporadically distributed within the lamina propria. In the Formula group, CD4⁺ T cells are dispersed below the epithelium. In the Formula/DSS group, CD4⁺ T cells are rarely observed in the superficial mucosa and the lamina propria, and most of the CD4⁺ T cells cluster inside the ILFs. In the DSS group, CD4⁺ T cells cluster in the submucosa. These results are consistent with FIG. 4, indicating that the prebiotic formula of the present disclosure may activate the innate CD4⁺ T cells responsible for monitoring the epithelial cells and the bacterial activity in the intestine. When epithelial harm occurs, CD4⁺ T cells migrate into the ILFs to play a role in transmitting signals and regulating the adaptive T cells.

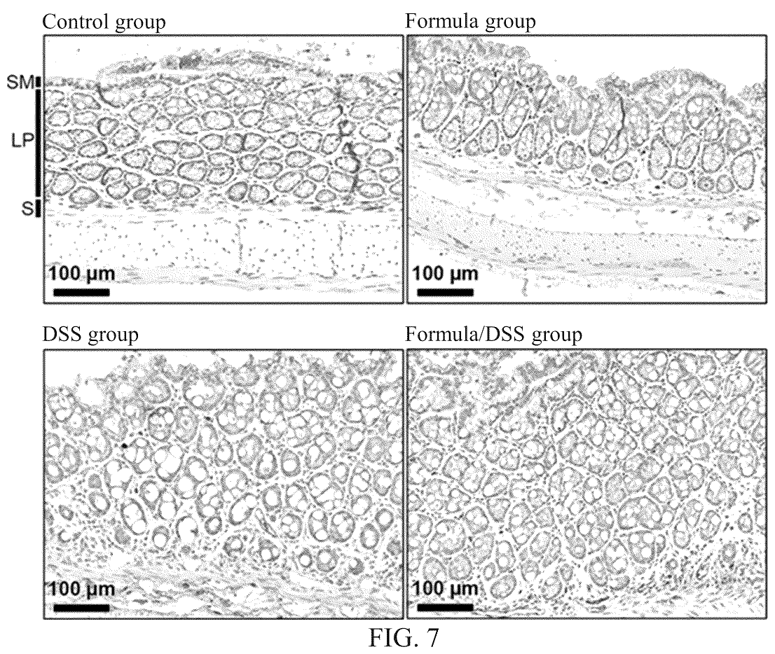

FIG. 7 shows the IHC staining images of IL-6 (presented by dark dots) from each experimental group. In the Control group, IL-6-secreting cells are sporadically distributed within the lamina propria. In the Formula group, IL-6-secreting cells are located below the epithelium. In the DSS group, IL-6-secreting cells cluster in the submucosa. In the Formula/DSS group, IL-6-secreting cells are scattered within the epithelium or crypts. These results are consistent with FIG. 5, indicating that the intraepithelial lymphocytes release IL-6 to affect the tight junctions of the epithelial cells, enhancing the mucosal barrier function.

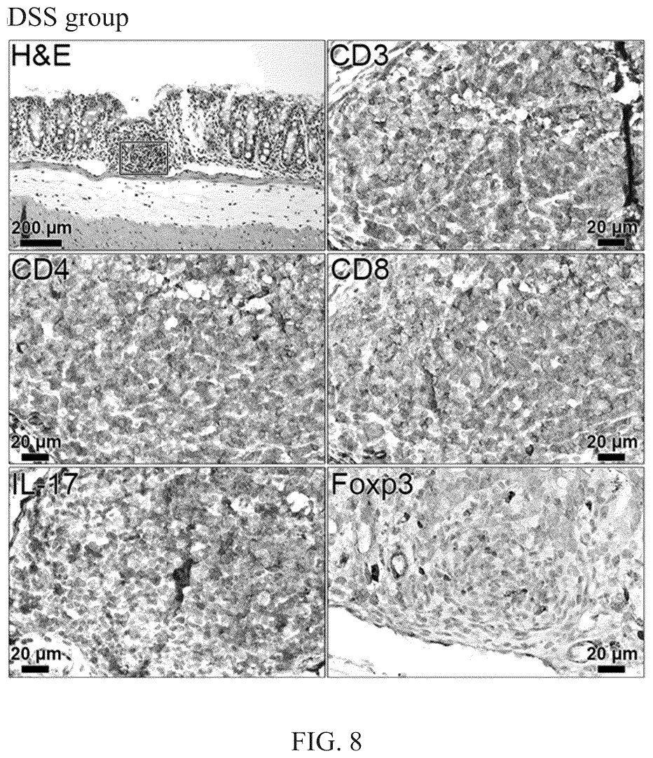

FIG. 8 shows the staining images of the expression of T cells within the ILFs in the DSS group. FIG. 9 shows the staining images of the expression of T cells within the ILFs in the Formula/DSS group. Except for the H&E staining images in FIGS. 8 and 9, all other staining images show the local magnifications of the staining images for CD3⁺ T cells, CD4+ T cells, CD8⁺ T cells, IL-17, and Foxp3 expression in the square box of the H&E staining images, wherein the black-blurry spots indicate expression, and the light-gray indicate non-expression. Compared to the H&E staining images in FIGS. 8 and 9, the DSS group presents a smaller cluster of lymphocytes, and the expressions of CD3⁺ T, CD4⁺ T, or CD8⁺ T cells are significantly fewer than that of the Formula/DSS group when the intestinal mucosal barrier is damaged by DSS. In Formula/DSS group, the ILFs are larger multiple times, which may be available for the differentiation, development and maturation of T cells, wherein the expressions of CD4⁺ T or CD8⁺ T cells increase significantly.

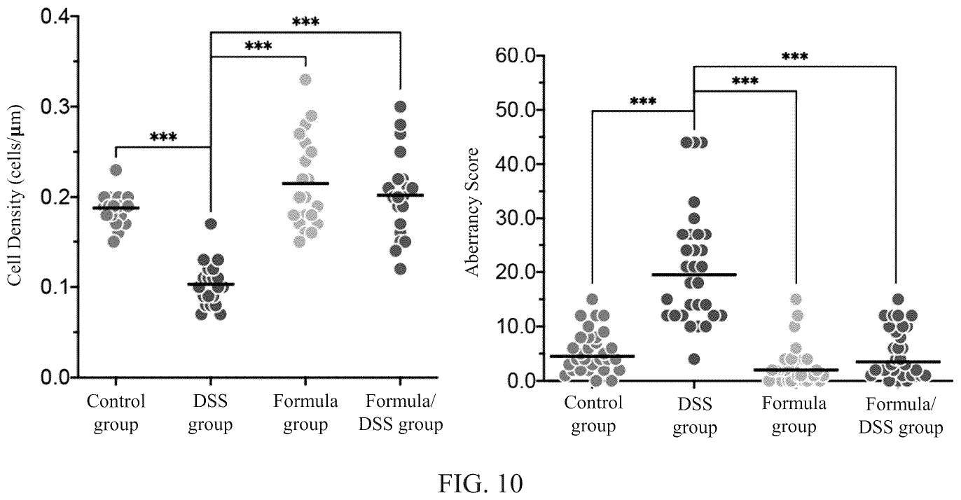

FIG. 10 shows the statistical analysis chart of the cell density of the epithelium and the aberrancy score of the intestinal mucosa from each experimental group. Compared to the DSS group, the Formula/DSS group exhibits a significant increase in the cell density of the epithelium and a significant decrease in aberrancy score. Furthermore, the Formula/DSS group demonstrate no significant difference from the Control and Formula groups, indicating that the Formula/DSS group has intact epithelium and healthy mucosal tissue compared to the DSS group.

The “aberrancy score” (AS) herein refers to the evaluation of tissue abnormalities or inflammation in the intestinal mucosa. The higher the score means the more aberrancy. The aberrancy score is calculated by the following equation, and the feature and grade for evaluation are shown in Table 2.

Aberrancy score=Inflammatory severity×Involvement+Inflammatory extent×Involvement+Epithelium regeneration×Involvement+Crypt damage×Involvement (30 sessions for each group are evaluated in blinded fashion by clinical pathology consultants.)

Table 2 shows histological scoring scheme for colitis.

| Feature | Grade | Criterion |
|---|---|---|
| Inflammatory | 0 | None |
| severity | 1 | Slight |
| | 2 | Moderate |
| | 3 | Severe |
| Inflammatory | 0 | None |
| extent | 1 | Mucosa |
| | 2 | Mucosa and submucosa |
| | 3 | Transmural |
| Epithelium | 0 | Complete regeneration |
| regeneration | 1 | Partially complete regeneration (with gland) |
| | 2 | Incomplete regeneration |
| | 3 | No tissue repair |
| Crypt damage | 0 | None |
| | 1 | Basal 1/3 damaged |
| | 2 | Basal 2/3 damaged |
| | 3 | Only surface epithelium intact |
| Involvement | 1 | 0-25% |
| | 2 | 25%-50% |
| | 3 | 50%-75% |
| | 4 | 75%-100% |

Collectively, it is evident that the Formula/DSS group has an intact intestinal mucosal barrier function compared to the DSS group, thereby avoiding damage to the intestinal epithelium and inflammation caused by DSS.

In addition, in the Control and Formula groups, the number of resident CD8⁺ T cells within the ILFs is far less than that of CD4⁺ T cells. Compared to the DSS group, the Formula/DSS group has significantly more CD8⁺ T cells in the ILFs, suggesting that the adaptive CD8⁺ T cells will accelerate proliferation and differentiation when the mucosa is damaged by DSS.

Moreover, compared to the Control group, the Formula group has a greater number and larger size of the ILFs in the intestinal mucosa, suggesting that the inducible microbiota stimulates the activation of the innate T cells and the aggregation of B cells.

Furthermore, since the Formula group has a greater number of more mature ILFs compared to the Control group, suggesting that the ILFs have better efficient of providing CD4⁺ T or CD8⁺ T during ICB therapy.

Prior to ICB therapy, continuous consumption of the disclosed prebiotic formula may facilitate the inducible microbiota to stimulate the ILFs mature, facilitating the differentiation, development and maturation of the adaptive CD4⁺ T, CD8⁺ T cells, which may assist ICB therapy. Moreover, the assessment of the type and relative abundance of the fecal microbiota of a patient ingesting the prebiotic formula of the present disclosure may serve as an indicator of an individual's response to ICB therapy.

What is claimed is:

1. A prebiotic formula, comprising:
glucomannan polysaccharides with a main distribution range of molecular weight at 0.6-107 kDa, said low molecular weight glucomannan polysaccharides, having β-1,4-D-glucopyranose units and β-1,4-D-mannopyranose units in backbone, and a portion of mannosyl units acetylated.

2. A dietary fiber, comprising the prebiotic formula according to claim 1.

3. A food, comprising the prebiotic formula according to claim 1.

4. A pharmaceutical composition, comprising the prebiotic formula according to claim 1.

5. An application of the prebiotic formula according to claim 1 in intestinal mucosa protection.

6. The application according to claim 5, wherein said prebiotic formula is fermented by inducible microbiota in intestine after ingestion, type of said inducible microbiota includes clostridial IV or XIV clusters or genera of Clostridium, Bacteroides, Alistipes, Bifidobacterium, Lactobacillus, Enterococcus or Akkermansia; and
said intestinal mucosa protection comprises, by said inducible microbiota, maintaining cell density of intestinal epithelium, reducing aberrancy of intestinal mucosa, maintaining or improving integrity of intestinal mucosal barrier, or increasing immune tolerance of intestinal mucosa.

7. An application of the prebiotic formula according to claim 1 in intestinal immunity modulation.

8. The application according to claim 7, wherein said prebiotic formula is fermented by inducible microbiota in intestine after ingestion, type of inducible microbiota includes clostridial IV or XIV clusters or genera of Clostridium, Bacteroides, Alistipes, Bifidobacterium, Lactobacillus, Enterococcus or Akkermansia; and
said intestinal immunity modulation comprises, by inducible microbiota, stimulating activation of innate immune cells, increasing number of isolated lymphoid follicles, or facilitating development and maturation of isolated lymphoid follicles.

9. An application of the prebiotic formula according to claim 1 in assisting immune checkpoint blockade therapy, wherein efficacy of therapy is improved, or intestinal toxicity in therapy is reduced or prevented.

10. The application according to claim 9, comprising achievement of increased number of isolated lymphoid follicles for providing mature adaptive CD4⁺ T, CD8⁺ T cells in therapy.
